(19) United States
(12) Patent Application Publication
(10) Pub. No.: US 2023/0382958 A1
(43) Pub. Date: Nov. 30, 2023

(54) FUSION PROTEIN FOR BACTERIAL SURFACE DISPLAY

(71) Applicant: Agency for Science, Technology and Research, Singapore (SG)

(72) Inventors: Richie Pei Kun Tay, Singapore (SG); Siak Wei OW, Singapore (SG)

(21) Appl. No.: 18/245,249

(22) PCT Filed: Sep. 10, 2021
(86) PCT No.: PCT/SG2021/050548

§ 371 Date: Mar. 14, 2023

(30) Foreign Application Priority Data
Sep. 14, 2020 (SG) 10202008968Y

(51) Int. Cl. C07K 14/335; C12N 9/02

(52) U.S. Cl. CPC C07K 14/335; C12N 9/0089; A61K 38/00

(57) ABSTRACT

The invention relates generally to the field of molecular biology and microbiology. Provided herein is an expression construct encoding a fusion protein. In one embodiment, there is provided an expression construct encoding a fusion protein, the expression construct comprising a nucleic acid encoding a first polypeptide for expression and a second polypeptide derived from the cell wall targeting region of a Lactobacillus plantarum Lys2 autolysin, wherein the first and second polypeptides are joined to form a fusion protein. In a specific embodiment, the second polypeptide comprises the CAD4a domain, which is a truncated domain containing only R1-R3 of the five-repeat SH3_5 domain of the Lys2 autolysin. Provided herein is also a fusion protein and the use of a fusion protein as defined herein.

### FIELD OF INVENTION

The invention relates generally to the field of molecular biology and microbiology. Provided herein is an expression construct encoding a fusion protein, a fusion protein and the use of a fusion protein as defined herein.

### BACKGROUND

Surface display of proteins on bacteria has various applications in biotechnology, including for biosensing; for screening protein and peptide libraries in drug discovery; for biocatalysis in food and chemical processing; and for the development of “living therapeutics” to treat gastrointestinal diseases. In biomedicine and food processing in particular, lactic acid bacteria (LAB) are advantageous hosts for protein display due to their food-grade status (“generally regarded as safe” per US Food and Drug Administration) and ease of culture. Further, many LAB strains from the Lactobacillus genus have potential health benefits (such as “probiotics”) that could complement any therapeutic use.

Lactic acid bacteria displaying heterologous proteins have been investigated for therapeutic use, especially for treating metabolic and gastrointestinal diseases. Protein display on LAB can also be used to develop bacterial vaccine vectors, wherein the innate immunogenicity of LAB obviates the need for adjuvants. Lactic acid bacteria displaying enzymes can be used as biocatalysts in industrial processes.

To enable surface display, a protein or peptide is typically fused to an anchoring domain that binds to the cell wall of a host bacterium. The anchoring domain can be covalently linked to a cell wall component, or it may bind non-covalently. LAB may be genetically engineered for surface display, which is often necessary for covalent anchoring, since protein expression, secretion and anchoring occur sequentially in a single cell. However, the use of genetically modified bacteria raises safety concerns and may encounter lower consumer acceptance and more severe regulatory scrutiny, especially when included in food or pharmaceutical preparations.

Accordingly, it is generally desirable to overcome or ameliorate one or more of the above mentioned difficulties.

### SUMMARY

Disclosed herein is an expression construct encoding a fusion protein, the expression construct comprising a nucleic acid encoding a first polypeptide for expression and a second polypeptide derived from the cell wall targeting region of a Lactobacillus plantarum Lys2 autolysin, wherein the first and second polypeptides are joined to form a fusion protein.

Disclosed herein is a recombinant host cell comprising an expression construct as defined herein.

Disclosed herein is a fusion protein encoded by an expression construct as defined herein.

Disclosed herein is a method of displaying a fusion protein on the surface of a lactic acid bacterium, the method comprising the steps of a) expressing a fusion protein as defined herein in a host cell, and b) immobilising the fusion protein onto the surface of a lactic acid bacterium.

Disclosed herein is a lactic acid bacterium comprising a fusion protein as defined herein attached to its surface.

Disclosed herein is a composition comprising a lactic acid bacterium as defined herein.

Disclosed herein is a lactic acid bacterium or composition as defined herein for use as a medicament.

Disclosed herein is a method of treating a condition or disease in a subject, the method comprising administering a lactic acid bacterium or composition of as defined herein to the subject.

Disclosed herein is a lactic acid bacterium or composition as defined herein for use in the treatment of a condition or disease in a subject.

Disclosed herein is the use of a lactic acid bacterium or composition as defined herein in the manufacture of a medicament for the treatment of a condition or disease in a subject.

Disclosed herein is a method of preventing a condition or disease in a subject, the method comprising administering a lactic acid bacterium or composition as defined herein to the subject.

Disclosed herein is a lactic acid bacterium or composition as defined herein for use in the prevention of a condition or disease in a subject.

Disclosed herein is the use of a lactic acid bacterium or composition as defined herein in the manufacture of a medicament for the prevention of a condition or disease in a subject.

### BRIEF DESCRIPTION OF DRAWINGS

Embodiments of the present invention are hereafter described, by way of non-limiting example only, with reference to the accompanying drawings in which:

FIG. 1 shows application of the CAD4a domain for immobilising a heterologous functional proteinaceous compound on the surface of lactic acid bacteria.

FIG. 2a shows the molecular organisation of the L. plantarum Lys2 protein and the derivation of the CAD4a anchoring domain SS, signal sequence; LC, low complexity region; Mur, muramidase; R1-R5, SH3_5 repeats. FIG. 2b shows an example of a protein construct of the invention containing the CAD4a domain.

FIG. 3 shows sequence domains of the constructs of the invention. The Lys2 cell wall-targeting region (CWT) is in bold. Inter-repeat regions are underlined. Spacers between the functional and anchoring domains are double-underlined. For the synthetic spacer, x is preferably 1-3 repeats.

FIG. 4 shows SDS-PAGE (top panels) and Western blot analyses (middle panels) of purified CAD4a protein constructs expressed in E. coli. The functional domains are Sirius (3a) and superoxide dismutase (SOD) (3b). Protein constructs are shown schematically in the bottom panels. 6H, His-tag.

FIG. 5a shows cell-associated fluorescence after mixing different lactic acid bacteria with Sirius-CAD4a. FIG. 5b shows fluorescence micrographs of L. fermentum with surface-bound Sirius-CAD4a. *p≤0.05; ***p≤0.001 vs. control.

FIG. 6 compares cell-associated fluorescence of L. fermentum in two stages of growth. ***p≤0.001 vs. control.

FIG. 7 shows the correlation between cell-associated fluorescence and the concentration of Sirius-CAD4a added to L. fermentum at a fixed cell density. The graph is used to determine the maximum amount of protein bound per cell.

FIG. 8 compares cell-associated fluorescence of L. fermentum pre-treated with 5 M LiCl (to remove surface proteins) or 10% v/v TCA (to remove cell wall lipoteichoic acids). ns, p>0.05; ***p≤0.001 vs. control.

FIG. 9 shows variations in cell-associated fluorescence on L. fermentum with different mixing conditions: temperature (9a), pH (9b), salt concentration (9c).

FIG. 10a shows enzyme activity of different SOD-CAD4a protein constructs with varying spacer lengths. FIG. 10b shows cell-associated SOD activity after mixing L. fermentum with SOD-CAD4a. ns, p>0.05; **p≤0.01 vs. control.

FIG. 11 shows residual cell-associated SOD activity after SOD-coated bacteria encapsulated in alginate-chitosan beads underwent simulated gastric digestion. **p≤0.01 vs. control.

FIG. 12a shows the design of Sirius-CAD4a variants containing different numbers of SH3_5 repeats. FIG. 12b is an SDS-PAGE gel of purified Sirius-CAD4a constructs. FIG. 12c shows cell-associated fluorescence after mixing L. fermentum at two growth stages with the Sirius-CAD4a variants. ns, p>0.05; ***p≤0.001 vs. control.

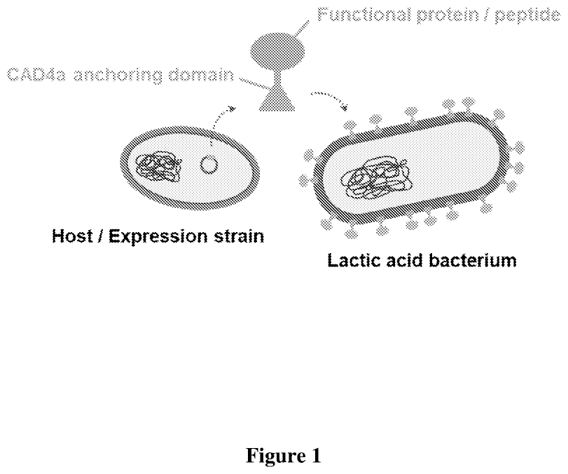

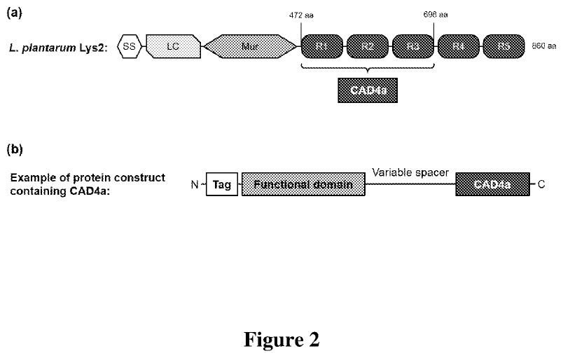

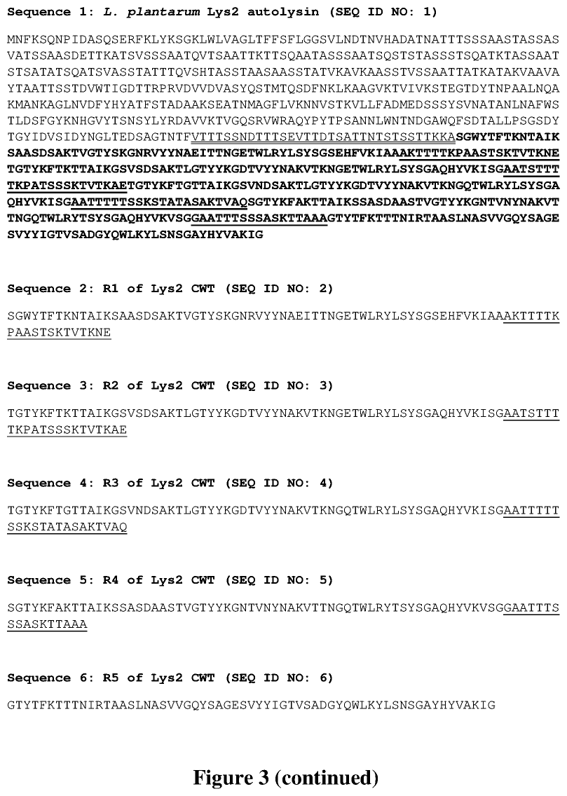

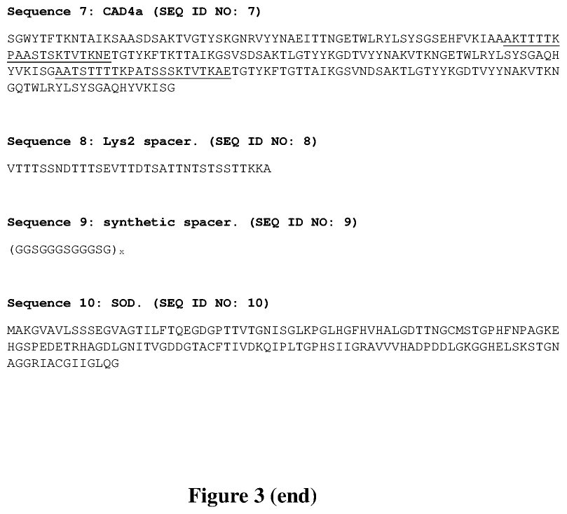

### DETAILED DESCRIPTION

The present specification teaches an expression construct encoding a fusion protein. Disclosed herein is an expression construct encoding a fusion protein, the expression construct comprising a nucleic acid encoding a first polypeptide for expression and a second polypeptide derived from the cell wall targeting region of a Lactobacillus autolysin, wherein the first and second polypeptides are joined to form a fusion protein.

In one embodiment, the second polypeptide is derived from the cell wall targeting region of a Lactobacillus plantarum Lys2 autolysin.

In one embodiment, the fusion protein is capable of being displayed on the surface of a lactic acid bacterium.

The term “polynucleotide” or “nucleic acid” are used interchangeably herein to refer to a polymer of nucleotides, which can be mRNA, RNA, cRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA.

The terms “protein” and “polypeptide” are used interchangeably and refer to any polymer of amino acids (dipeptide or greater) linked through peptide bonds or modified peptide bonds. Polypeptides of less than about 10-20 amino acid residues are commonly referred to as “peptides.” The polypeptides of the invention may comprise non-peptidic components, such as carbohydrate groups. Carbohydrates and other non-peptidic substituents may be added to a polypeptide by the cell in which the polypeptide is produced, and will vary with the type of cell. Polypeptides are defined herein, in terms of their amino acid backbone structures; substituents such as carbohydrate groups are generally not specified, but may be present nonetheless.

A “fusion protein” is a hybrid protein expressed by a nucleic acid molecule comprising nucleotide sequences of at least two genes. In this way, a fusion protein comprises as least two amino acid sequences that are not associated with each other in nature.

The term “derived from” as used herein in reference to a nucleic acid means that at least a portion of the nucleic acid (e.g. gene, gene portion, regulatory element, polypeptide) is also present in (i.e. or was copied from) the biological source that the nucleic acid was derived from. The derived nucleic acid may be constructed in any way that provides the desired sequence, including the derivative portion. For example, nucleic acid may be obtained directly from the biological source, using restriction enzymes or other tools of molecular biology, or by amplifying from the biological source (e.g. by polymerase chain reaction), or by a technique such as chemical synthesis. While in many instances a nucleic acid derived from a biological source is not directly obtained from the source, its sequence and/or characteristics are substantially the same as a portion of sequence from the biological source.

The fusion protein as defined herein may comprise one or more conservative amino acid substitutions.

A “conservative amino acid substitution” is to be understood as meaning a substitution in which the amino acid residue is replaced with an amino acid residue having a similar side chain Families of amino acid residues having similar side chains have been defined in the art, which can be generally sub-classified as shown in the table “Amino Acid Classification”, below:

Amino Acid Sub-Classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, |
| | Histidine |
| Small | Glycine, Serine, Alanine, Threonine, Proline |
| Polar/neutral | Asparagine, Histidine, Glutamine, Cysteine, |
| | Serine, Threonine |
| Polar/large | Asparagine, Glutamine |
| Hydrophobic | Tyrosine, Valine, Isoleucine, Leucine, Methionine, |
| | Phenylalanine, Tryptophan |
| Aromatic | Tryptophan, Tyrosine, Phenylalanine |
| Residues that | Glycine and Proline |
| influence chain | |
| orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional polypeptide can readily be determined by assaying its activity.

Conservative substitutions are also shown in the table below (EXEMPLARY AND PREFERRED AMINO ACID SUBSTITUTIONS) Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants can be screened for their ability to bind specifically to NGF using methods known to persons skilled in the art, including those methods described elsewhere herein.

Exemplary and Preferred Amino Acid Substitutions

| Original | | Preferred |
|---|---|---|
| Residue | Exemplary Substitutions | Substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

The term “construct” refers to a recombinant genetic molecule including one or more isolated nucleic acid sequences from different sources. Thus, constructs are chimeric molecules in which two or more nucleic acid sequences of different origin are assembled into a single nucleic acid molecule and include any construct that contains (1) nucleic acid sequences, including regulatory and coding sequences that are not found together in nature (i.e., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative constructs include any recombinant nucleic acid molecule such as a plasmid, cosmid, virus, autonomously replicating polynucleotide molecule, phage, or linear or circular single stranded or double stranded DNA or RNA nucleic acid molecule, derived from any source, capable of genomic integration or autonomous replication, comprising a nucleic acid molecule where one or more nucleic acid molecules have been operably linked. Constructs of the present invention will generally include the necessary elements to direct expression of a nucleic acid sequence of interest that is also contained in the construct. Such elements may include control elements or regulatory sequences such as a promoter that is operably linked to (so as to direct transcription of) the nucleic acid sequence of interest, and often includes a polyadenylation sequence as well. Within certain embodiments of the invention, the construct may be contained within a vector. In addition to the components of the construct, the vector may include, for example, one or more selectable markers, one or more origins of replication, such as prokaryotic and eukaryotic origins, at least one multiple cloning site, and/or elements to facilitate stable integration of the construct into the genome of a host cell. Two or more constructs can be contained within a single nucleic acid molecule, such as a single vector, or can be containing within two or more separate nucleic acid molecules, such as two or more separate vectors. An “expression construct” generally includes at least a control sequence operably linked to a nucleotide sequence of interest. In this manner, for example, promoters in operable connection with the nucleotide sequences to be expressed are provided in expression constructs for expression in an organism or part thereof including a host cell. For the practice of the present invention, conventional compositions and methods for preparing and using constructs and host cells are well known to one skilled in the art, see for example, Molecular Cloning: A Laboratory Manual, 3rd edition Volumes 1, 2, and 3. J. F. Sambrook, D. W. Russell, and N. Irwin, Cold Spring Harbor Laboratory Press, 2000.

The term “encode” or “encoding” includes reference to nucleotides and/or amino acids that correspond to other nucleotides or amino acids in the transcriptional and/or translational sense.

By “control element”, “control sequence”, “regulatory sequence” and the like, as used herein, mean a nucleic acid sequence (e.g., DNA) necessary for expression of an operably linked coding sequence in a particular host cell. The control sequences that are suitable for prokaryotic cells for example, include a promoter, and optionally a cis-acting sequence such as an operator sequence and a ribosome binding site. Control sequences that are suitable for eukaryotic cells include transcriptional control sequences such as promoters, polyadenylation signals, transcriptional enhancers, translational control sequences such as translational enhancers and internal ribosome binding sites (IRES), nucleic acid sequences that modulate mRNA stability, as well as targeting sequences that target a product encoded by a transcribed polynucleotide to an intracellular compartment within a cell or to the extracellular environment.

The term “expression” refers to the cellular processes involved in producing RNA and proteins and as appropriate, secreting proteins, including where applicable, but not limited to, for example, transcription, transcript processing, translation and protein folding, modification and processing.

By “linker” is meant a molecule or group of molecules (such as a monomer or polymer) that connects two molecules and often serves to place the two molecules in a desirable configuration. In specific embodiments, a “linker” refers to an amino acid sequence that connects the first and second polypeptides and may provide a spacer function compatible with the spacing of the first and second polypeptides to allow the first polypeptide to be displayed on the surface of a lactic acid bacterium. In other embodiments, the “linker” refers to a chemical linker.

In one embodiment, the first and second polypeptides are separated by a linker polypeptide. The linker polypeptide can be of any length. The linker polypeptide can be about 10-50 amino acids, or about 10-30 amino acids, in length. The linker length and property may be arbitrary and customised to the protein of interest. Typically, “linkers” are peptides chosen or designed to be unstructured and flexible. For instance, one can choose amino acids that form no particular secondary structure. Or, amino acids can be chosen so that they do not form a stable tertiary structure. Or, the amino acid linkers may form a random coil. Such linkers include, but are not limited to, synthetic peptides rich in Gly, Ser. Thr, Gln, Glu or further amino acids that are frequently associated with unstructured regions in natural proteins.

The linker polypeptide may, for example, be VTTTSSNDTTTSEVTTDTSATTNTSTSSTTKKA (SEQ ID NO:8). The linker polypeptide may also be (GGSGGGSGGGSG)ₓ (SEQ ID NO:9), where X is an integer, such as 1, 2, 3 or more. Other examples include (GS)₅ or (GS)₁₀.

In an alternative embodiment, the first and second polypeptides are separated by a chemical linker.

In one embodiment, the first polypeptide is positioned upstream of the second polypeptide. In one embodiment, the first polypeptide is positioned downstream of the second polypeptide.

The second polypeptide may be derived from the cell wall targeting region of a Lactobacillus autolysin. In one embodiment, the Lactobacillus autolysin comprises a cell wall targeting region. In one embodiment, the Lactobacillus autolysin is a Lactobacillus plantarum Lys2 autolysin. The Lactobacillus plantarum Lys2 autolysin may be one having the GenBank Accession Number of CCC80137.1. The Lactobacillus autolysin may comprise or consist of an amino acid sequence having at least 70% (including at least 71% to 99% and all integer percentages therebetween) sequence identity to an amino acid sequence of SEQ ID NO: 1.

In one embodiment, the second polypeptide is capable of anchoring the fusion protein onto the surface of a bacterium, such as a lactic acid bacterium. In one embodiment, the second polypeptide comprises or consists of a SH3 type 5 motif polypeptide. In one embodiment, the second polypeptide comprises or consists of an amino acid sequence having at least 70% (including at least 71% to 99% and all integer percentages therebetween) sequence identity to an amino acid sequence of SEQ ID NO: 2, SEQ ID NO: 3, SEQ ID NO: 4, SEQ ID NO: 5 and SEQ ID NO: 6, or combinations thereof (such as SEQ ID NOs: 2 and 3, SEQ ID Nos 2, 3, and 4, SEQ ID Nos 2, 3, 4 and 5, SEQ ID Nos: 2, 3, 4, 5, and 6, SEQ ID NOs: 3 and 4, SEQ ID NOS: 3, 4 and 5, SEQ ID NOs: 3, 4, 5 and 6, SEQ ID NOs: 4 and 5, SEQ ID NOs: 4, 5 and 6, SEQ ID NOs: 5 and 6).

In one embodiment, the second polypeptide comprises or consists of an amino acid sequence having at least 70% (including at least 71% to 99% and all integer percentages therebetween) sequence identity to an amino acid sequence of SEQ ID NO: 7.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G and I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

Terms used to describe sequence relationships between two or more polynucleotides or polypeptides include “reference sequence,” “comparison window”, “sequence identity,” “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res.25:3389. A detailed discussion of sequence analysis can be found in Unit 19.3 of Ausubel et al., “Current Protocols in Molecular Biology”, John Wiley & Sons Inc, 1994-1998, Chapter 15.

The first polypeptide may be any polypeptide of biological interest (such as a biologically active or therapeutic polypeptide or an antigenic polypeptide). The first polypeptide can be an anti-viral polypeptide, an anti-bacterial polypeptide, an anti-fungal polypeptide, or a polypeptide that binds to viruses, bacteria or fungi, including antibodies, antibody fragments or single-chain antibodies. The first polypeptide can be an antigenic polypeptide from viruses, bacteria or fungi.

Examples of viruses include, but are not limited to, Retroviridae (e.g., human immunodeficiency viruses, such as HIV-1 (also referred to as HTLV-III, LAV or HTLV-III/LAV, or HIV-III); and other isolates, such as HIV-LP; Picornaviridae (e.g., polio viruses, hepatitis A virus; enteroviruses, human coxsackie viruses, rhinoviruses, echoviruses); Caliciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses, including SARS-CoV-2 virus); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae (e.g., reoviruses, orbiviurses and rotaviruses); Bimaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes viruses); Poxyiridae (variola viruses, vaccinia viruses, pox viruses); and Iridoviridae (e.g., African swine fever virus); Hepatitis C virus; and unclassified viruses (e.g., the agent of delta hepatitis (thought to be a defective satellite of hepatitis B virus); Norwalk and related viruses, and astroviruses).

Examples of bacteria include, but are not limited to, Helicobacter pylorus, Borrelia burgdorferi, Legionella pneumophila, Mycobacteria sps (e.g., M tuberculosis, M avium, M. intracellulare, M. kansasii, M gordonae), Staphylococcus aureus, Neisseria gonorrhoeae, Neisseria meningitidis, Listeria monocytogenes, Streptococcus pyro genes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, pathogenic strains of Escherichia coli, Streptobacillus monilifonnis, Treponema pallidium, Treponema pertenue, Leptospira, and Actinomyces israelii.

Examples of fungi include, but are not limited to, Cryptococcus neoformans, Histoplasma capsulatum, Coccidioides immitis, Blastomyces dermatitidis, Chlamydia trachomatis and Candida albicans.

As used herein, the term “antigen” and its grammatically equivalents expressions (e.g., “antigenic”) refer to a polypeptide, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins.

A “therapeutic protein” or “therapeutic polypeptide” includes a protein or polypeptide that the host does not produce but is in need of; a protein that the host does not normally produce, but which has a therapeutic activity; a protein that the host produces, but produces in inadequate amounts; a protein that the host produces but in a form which is inactive, or which has reduced activity compared with an activity normally associated with the protein; or a protein that the host produces in adequate amounts and with normal activity associated with that protein. Therapeutic proteins include naturally-occurring proteins, and recombinant proteins whose amino acid sequences differ from a naturally-occurring counterpart protein, which recombinant proteins have substantially the same, an altered activity, or enhanced activity relative to a naturally-occurring protein. Proteins that have therapeutic activity include, but are not limited to, cytokines, including, but not limited to, interleukins, endothelin, colony stimulating factors, tumor necrosis factor, and interferons; hormones, including, but not limited to, a growth hormone, insulin; growth factors, including, but not limited to human growth factor, insulin-like growth factor; bioactive peptides; trophins; neurotrophins; soluble forms of a membrane protein including, but not limited to, soluble CD4; enzymes; regulatory proteins; structural proteins; clotting factors, including, but not limited to, factor XIII; erythropoietin; tissue plasminogen activator; etc.

The first polypeptide may be an enzyme. The enzyme may, for example, be superoxide dismutase (SOD), cellulase, amylase, lipase or protease.

In one embodiment, the first polypeptide is superoxide dismutase (SOD). In one embodiment, the first polypeptide may consist or comprise an amino acid having at least 70% (including at least 71% to 99% and all integer percentages therebetween) sequence identity to an amino acid sequence of SEQ ID NO: 10.

Disclosed herein is a recombinant host cell comprising an expression construct as defined herein. In one embodiment, the host cell is Escherichia coli or Lactococcus lactis.

Introduction of the expression construct to a host cell may be effected by any method known to those skilled in the art. For example, the expression construct may be introduced by transformation, electroporation or transfection.

Disclosed herein is a recombinant host cell encoding a fusion protein comprising a first polypeptide for expression and a second polypeptide derived from the cell wall targeting region of a Lactobacillus autolysin, wherein the first and second polypeptides are joined to form a fusion protein.

The terms “host”, “host cell”, “host cell line” and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the fusion proteins of the present invention. Host cells include cultured cells, e.g., bacterial cells, such as Escherichia (e.g. Escherichia coli), Lactococcus (e.g. Lactococcus lactis), Bacillus, Lactobacillus, Pseudomonas, Streptomyces, coryneform bacteria, and halophilic bacteria, mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

Disclosed herein is a fusion protein encoded by an expression construct as defined herein. In one embodiment, the fusion protein is a recombinant fusion protein.

In one embodiment is a nucleic acid encoding a fusion protein comprising a first polypeptide for expression and a second polypeptide derived from the cell wall targeting region of a Lactobacillus autolysin, wherein the first and second polypeptides are joined to form a fusion protein.

Disclosed herein is a fusion protein comprising a first polypeptide for expression and a second polypeptide derived from the cell wall targeting region of a Lactobacillus autolysin, wherein the first and second polypeptides are joined to form a fusion protein.

In one embodiment, there is provided the use of a fusion protein as defined herein for display on the surface of a lactic acid bacterium. The fusion protein may be a heterologous fusion protein as defined herein.

In one embodiment, there is provided a system for the expression of a heterologous protein on the surface of a lactic acid bacterium.

Disclosed herein is a method of displaying a fusion protein on the surface of a lactic acid bacterium, the method comprising the steps of a) expressing a fusion protein as defined herein in a host cell, and b) immobilising the fusion protein onto the surface of a lactic acid bacterium.

In one embodiment, the method comprises isolating and/or purifying the fusion protein prior to step b).

The immobilisation may be carried out in the temperature range of 25 to 37° C. If live bacteria are used as hosts, the immobilisation temperature may be conducted at the temperature range for bacterial culture. The immobilisation may be carried out in the pH range of 5 to 7. If live bacteria are used as hosts, the immobilisation pH may be carried out within the pH range for bacterial culture. The immobilisation may be carried out at physiological salt concentrations, such as at about 50 to 200 mM NaCl.

The method may comprise pre-treatment of the lactic acid bacterium so as to increase the amount of fusion protein that is bound to the cell surface. For example, pre-treatment of bacteria with detergents, organic solvents, acids and salts may selectively remove components of the cell wall without affecting the peptidoglycans. This may expose more sites on the cell wall for binding to the fusion protein.

In one embodiment, the lactic acid bacterium is a Lactococcus or Lactobacillus bacterium. The lactic acid bacterium may, for example, be Lactococcus lactis, Lactobacillus casei, Lactobacillus fermentum, Lactobacillus plantarum and Lactobacillus rhamnosus. In one embodiment, the lactic acid bacterium is Lactobacillus fermentum.

In one embodiment, a non-viable cell inactivated by heat, UV, gamma irradiation or chemical treatment is used in place of the lactic acid bacterium.

Disclosed herein is a lactic acid bacterium comprising a fusion protein as defined herein attached to its surface. The fusion protein may be a heterologous fusion protein as defined herein. The fusion protein may be immobilised on the surface of the lactic acid bacterium.

The lactic acid bacterium may be live, inactivated, non-viable, non-replicating or dead.

Disclosed herein is a composition comprising a lactic acid bacterium as defined herein. The composition may be a food composition, a pharmaceutical composition or an immunomodulatory composition.

Where the intended use is to elicit or increase an immune response, the composition is referred to as an “immunogenic” or “immunomodulating” composition. Such compositions include preventative compositions and therapeutic compositions. An immunomodulating composition of the present invention may therefore be administered to a recipient for prophylactic, ameliorative, palliative, or therapeutic purposes.

The present disclosure further provides compositions, including food compositions, pharmaceutical compositions or immunomodulatory compositions comprising a lactic acid bacterium as defined herein. Representative compositions may include a buffer, and may also include other substances appropriate to the intended use.

Those skilled in the art can readily select an appropriate buffer, a wide variety of which are known in the art, suitable for an intended use. In some instances, the composition can comprise a pharmaceutically acceptable excipient, a variety of which are known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7.sup.th ed., Lippincott, Williams, & Wilkins; and Handbook of Pharmaceutical Excipients (2000) A. H. Kibbe et al., eds., 3.sup.rd ed. Amer. Pharmaceutical Assoc.

In one embodiment, the composition is a food composition. To prevent protein detachment, bacteria with surface-bound protein may be mixed with a food matrix or food-grade biomaterial that buffers against environmental changes. Food matrices include dairy products and fermented foods. Food-grade biomaterials may be edible polymers and enteric capsules. The food composition may be milk, yoghurt, curd, cheese, fermented milks, milk based fermented products, ice cream, fermented cereal based products, milk based powders, infant formulae or, in case of animals, pet food.

In one embodiment, the lactic acid bacterium is encapsulated. The lactic acid bacterium may, for example, be encapsulated in chitosan-coated alginate beads.

In one embodiment, the composition is a freeze-dried or hydrated composition. The lactic acid bacterium may be used in a hydrated or dried form. The drying process may be any system that preserves the integrity of the bacterial cell wall and the bound protein, including freeze-drying and spray-drying.

In one embodiment, the composition comprises an immobilised fusion protein. The fusion protein may be immobilized within a matrix in the composition. The host bacterium with surface-immobilised fusion protein may be used in an industrial process for bioconversion.

Use of a lactic acid bacterium of composition as defined herein as a probiotic.

Disclosed herein is a lactic acid bacterium or composition as defined herein for use as a medicament. The medicament may, for example, be a vaccine (such as a mucosal vaccine).

Disclosed herein is a method of treating a condition or disease in a subject, the method comprising administering a lactic acid bacterium or composition as defined herein to the subject.

In one embodiment, the method comprises administering a therapeutically effective amount of a lactic acid bacterium or composition as defined herein to the subject.

As used herein the term “therapeutically effective amount” includes within its meaning a non-toxic but sufficient amount of a lactic acid bacterium or composition to provide the desired therapeutic effect. The exact amount required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

The terms “treating”, “treatment” and the like, are used interchangeably herein to mean relieving, reducing, alleviating, ameliorating or otherwise inhibiting the condition, including one or more symptoms of the condition. The terms “prevent”, “preventing”, “prophylaxis”, “prophylactic”, “preventative” and the like are used interchangeably herein to mean preventing or delaying the onset of the condition, or the risk of developing the condition.

The terms “treating”, “treatment” and the like also include relieving, reducing, alleviating, ameliorating or otherwise inhibiting the effects of the condition for at least a period of time. It is also to be understood that terms “treating”, “treatment” and the like do not imply that the condition, or a symptom thereof, is permanently relieved, reduced, alleviated, ameliorated or otherwise inhibited and therefore also encompasses the temporary relief, reduction, alleviation, amelioration or otherwise inhibition of the condition, or of a symptom thereof.

The terms “subject”, “patient”, “host” or “individual” used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including primates (e.g., humans, monkeys and apes, and includes species of monkeys such as from the genus Macaca (e.g., cynomolgus monkeys such as Macaca fascicularis, and/or Rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. In one embodiment, the subject is a human subject.

Disclosed herein is a lactic acid bacterium or composition as defined herein for use in the treatment of a condition or disease in a subject.

Disclosed herein is the use of a lactic acid bacterium or composition as defined herein in the manufacture of a medicament for the treatment of a condition or disease in a subject.

In one embodiment, the condition or disease is inflammatory bowel disease (such as chronic colitis, Crohn's disease or ulcerative colitis), oral mucositis, cancer, or an allergy. In one embodiment, the condition or disease is a gut condition or disease. In one embodiment, the condition or disease is an oral disease or condition.

Disclosed herein is a method of preventing a condition or disease in a subject, the method comprising administering a lactic acid bacterium or composition as defined herein to the subject.

Disclosed herein is a lactic acid bacterium or composition as defined herein for use in the prevention of a condition or disease in a subject.

Disclosed herein is the use of a lactic acid bacterium or composition as defined herein in the manufacture of a medicament for the prevention of a condition or disease in a subject.

In one embodiment, the condition or disease is a pathogenic infection. The pathogenic infection may be an infection by bacteria, viruses, fungi, protozoa or prion.

Disclosed herein is a method of modulating an immune response in a subject, the method comprising administering a lactic acid bacterium or composition as defined herein to the subject.

An “immune response” as used herein, refers to a response by the immune system of a subject. For example, an immune response may be to an antigen/immunogen that the subject's immune system recognizes as foreign (e.g., non-self-antigens) or self (e.g., self-antigens recognized as foreign) Immune responses may be humoral, involving production of immunoglobulins or antibodies, or cellular, involving various types of B and T lymphocytes, dendritic cells, macrophages, antigen presenting cells and the like, or both. Immune responses may also involve the production or elaboration of various effector molecules such as cytokines. The term “immune response” encompasses immunogenic responses that cause, activate, elicit, stimulate, or induce an immune response against a particular antigen (e.g., an antigen of a pathogenic organism) or organism (e.g., a pathogenic microorganism) in a subject, as well as immunosuppressive or tolerogenic immune responses that inhibit, suppress, diminish or eliminate an immune response, or render the immune system unresponsive, or delay the occurrence or onset of an immune response, to an allergen, or to a self-antigen or a cell, tissue or organ that expresses such an antigen.

Disclosed herein is a lactic acid bacterium or composition as defined herein for use in modulating an immune response in a subject.

Disclosed herein is the use of a lactic acid bacterium or composition as defined herein in the manufacture of a medicament for modulating an immune response in a subject.

Disclosed herein is a kit comprising an expression construct, fusion protein or lactic acid bacterium as defined herein.

The articles “a” and “an” are used herein to refer to one or to more than one (i.e., to at least one) of the grammatical object of the article. By way of example, “an expression construct” means one expression construct or more than one expression construct.

By “about” is meant a quantity, level, value, number, frequency, percentage, dimension, size, amount, weight, position or length that varies by as much 10, 9, 8, 7, 6, 5, 4, 3, 2 or 1% to a reference quantity, level, value, number, frequency, percentage, dimension, size, amount, weight, position or length.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### EXAMPLES

### Example 1

Design of CAD4a gene constructs and expression of protein constructs A search of Pfam database revealed 87 SH3_5 homologs across the Lactobacillus and Lactococcus genera and their phages. A previously uncharacterized cell anchoring domain in Lys2 (lp_3093), a muramidase from L. plantarum str. WCFS1, was selected. The full-length muramidase is an 860-amino acid protein with an expected molecular weight of 84 kDa, with five SH3_5 repeats at its C-terminus spanning residues 471 and 860; each repeat is 60 residues.

To assess the surface display potential of this five-repeat SH3_5 domain, the Sirius blue fluorescent protein was used as a reporter. Sirius was chosen for its photostability at low pH (pKa<3), which was advantageous in later simulated digestion experiments. The five-repeat domain was cloned downstream of Sirius in the pET22b plasmid, but the fusion protein was insoluble and difficult to purify. A truncated domain containing only R1-R3 (henceforth called CAD4a) gave more tractable expression, and the Sirius conjugate (Si-CAD4a) was easily detected by SDS-PAGE and Western blot against the N-terminal His-tag. There was some protein degradation during expression, but the bulk of the soluble fraction was the full-length protein.

Bacterial Strains, Culture Conditions and Plasmid Assembly

The bacteria strains and plasmids used in this example are listed in Table 1. Cloning was performed in E. coli Turbo and proteins were expressed in E. coli BL21(DE3) as detailed in the next section. E. coli was selected on LB agar supplemented with 100 μg/ml carbenicillin. Lactic acid bacteria were grown in static, unaerated MRS broth (Sigma, USA) at 37° C.

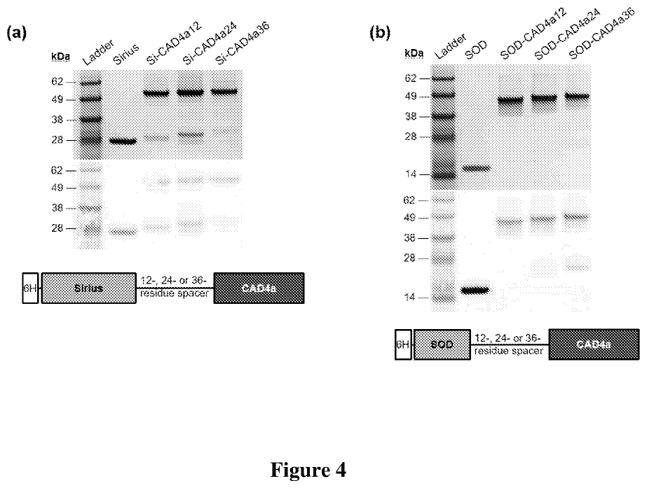

Table 2 lists the plasmids, primers and synthetic gene fragments used in this study. Primers and gene fragments were synthesised by Integrated DNA Technologies (USA). Gibson assembly was used to construct all plasmids. Protein constructs are illustrated schematically in the bottom panels of FIGS. 4a and 4b. Briefly, proteins contained an N-terminal 6×His tag and a C-terminal CAD4a domain Spacers between the functional and CAD4a domains were 12-, 24- or 36-residues in length, with sequence given in Sequence 9.

For the Sirius constructs, the pET22b plasmid was first linearised with primers F1 and R1, and assembled with fragment G10 to give pET22b-Sirius. pET22b-Sirius was linearised with primers F1 and either R7 or R6, to give pET22b-Si-CAD4a12 or —Si— CAD4a24 respectively, after assembly with gene fragment G5. CAD4a was subcloned from pET22b-Si-CAD4a24 using primers F15 and R5, then assembled with pET-Sirius linearised with F1 and R26, to give pET22b-Si-CAD4a36.

For the SOD constructs, G29 was amplified with primers F18 and R37, and pET22b-Sirius linearised with primers F1 and R4; both fragments were then assembled to give pET22b-SOD. pET22b-SOD was linearised with primers F1 and either R24 or R25, to give pET22b-SOD-CAD4a12 or -SOD-CAD4a24 respectively, after assembly with gene fragment G5. CAD4a was subcloned from pET22b-Si-CAD4a24 with primers F15 and R5, then assembled with pET-SOD linearised with F1 and R27, to give pET22b-SOD-CAD4a36.

Protein Expression

Overnight E. coli cultures were diluted 1:100 in Terrific Broth and grown to optical density OD₆₀₀˜0.8. At that point, the temperature was reduced to 20° C. and sorbitol was added to a concentration of 0.4 M. Sorbitol was added only for expression of CAD4a protein conjugates, to reduce protein aggregation. Expression was induced with 0.2 mM IPTG and allowed to proceed for 6 hr at 20° C. Cells were then pelleted at 4000 g for 10 min, resuspended in Tris buffer (50 mM Tris, 0.3 M NaCl, pH 8), and subjected to one freeze-thaw cycle before lysis on ice with a probe sonicator (Microson XL2000, 10 s ON, 10 s OFF, 8 cycles). The lysate was pelleted at 12,000 g for 30 min at 4° C., and separated on Ni-NTA resin (Qiagen, USA) in a PD-10 column. His-tagged protein was eluted with 150 mM imidazole, then concentrated and buffer-exchanged into 1×PBS (pH 7.4) and stored at 4° C. until use.

SDS-PAGE and Western Blot

Protein concentrations were determined using Bradford reagent (Biorad). Protein samples were analysed on NuPAGE 4-12% Bis-Tris gels (Life Technologies), following manufacturer's protocols. Gels were stained with InstantBlue (Expedeon) or transferred onto nitrocellulose membranes using the semi-dry method at 20 V for 20 min (Trans-Blot, Bio-Rad). The membrane was washed with TBST (1×TBS, 0.1% Tween 20), blocked with 5% w/v non-fat dry milk in TBST for 1 hr at room temperature, then exposed to a 1:10,000 dilution of HRP-conjugated anti-His antibody (Merck) for 1 hr at room temperature before detection with Clarity Western ECL Blotting Substrate (Bio-Rad) using the manufacturer's protocol. Gel images were acquired on a ChemiDoc MP imaging system (Bio-Rad), and blots were imaged on an ImageQuant LAS 500 imager (GE Healthcare). SDS-PAGE and Western blot analyses of all purified proteins are given in FIG. 4.

TABLE 1
Bacterial strains and plasmids used in this example.

| | |
|---|---|
| Feature | Source |

Strains

| | | |
|---|---|---|
| E. coli Turbo | Cloning host, TG1 derivative | NEB |
| | glnV44 thi-1 Δ(lac-proAB) galE15 galK16 | |
| | R(zgb-210::Tn10)Tet^{S} endA1 fhuA2 Δ(mcrB- | |
| | hsdSM)5, (r_{K}⁻m_{K}⁻) F′[traD36 proAB⁺ lacI^{q} | |
| | lacZΔM15] | |
| E. coli BL21(DE3) | Expression host, E. coli str. B | Thermo |
| | F⁻ ompT gal dcm lon hsdS_{B}(r_{B}⁻M_{B}⁻) λ(DE3 | Fisher |
| | [lacI lacUV5-T7p07 ind1 sam7 nin5]) | |
| | [malB⁺]_{K-12}(λ^{S}) | |
| Lactococcus lactis NZ9000 | Binding host, MG1363 derivative | MoBiTec |
| | pepN::nisRK | |
| Lactobacillus casei 393 | Binding host, wild type | ATCC |
| Lactobacillus fermentum | Binding host, wild type | ATCC |
| 14931 | | |
| Lactobacillus plantarum | Binding host, wild type | ATCC |
| 8014 | | |
| Lactobacillus rhamnosus GG | Binding host, wild type | Lesaffre |

Plasmids

| | | |
|---|---|---|
| pET22b | P_{T7}, Amp^{R}, lacI gene, N-terminal pelB seq | Novagen |
| pET22b-Sirius | His-tagged Sirius | This study |
| pET22b-Sirius-CAD4a12 | 12-residue spacer between Sirius and CAD4a | This study |
| pET22b-Sirius-CAD4a24 | 24-residue spacer between Sirius and CAD4a | This study |
| pET22b-Sirius-CAD4a36 | 36-residue spacer between Sirius and CAD4a | This study |
| pET22b-SOD | His-tagged SOD | This study |
| pET22b-SOD-CAD4a12 | 12-residue spacer between SOD and CAD4a | This study |
| pET22b-SOD-CAD4a24 | 24-residue spacer between SOD and CAD4a | This study |
| pET22b-SOD-CAD4a36 | 36-residue spacer between SOD and CAD4a | This study |

TABLE 2
Primers and synthetic gene fragments used in this example.

Sequence

Primers

| | |
|---|---|
| F1 | 5′-TGAGATCCGGCTGCTAACAAAGCCCGAAAG (SEQ ID NO: 11) |
| F15 | 5′-GGCGGGTCCGGCGGTGGAAGTGGCGGTGGTAGTGGCGGAGGTTCGG |
| | GGGGTGGCAGCG (SEQ ID NO: 12) |
| F18 | 5′-CCACGGTGGGTCTGGGGGGGGATCCGGCATGGCAAAGGGAGTGGCG |
| | GTACTGT (SEQ ID NO: 13) |
| R1 | 5′-GCCTTGGTTTTCTAATTTTGGTTCAAAGAAAGCT (SEQ ID NO: 14) |
| R4 | 5′-GCCGGATCCCCCCCCAGACCCAC (SEQ ID NO: 15) |
| R5 | 5′-CTTTCGGGCTTTGTTAGCAGCCGGATCTCA (SEQ ID NO: 16) |
| R6 | 5′-GCCTGACCCACCACCACTCCCGCCGCCGGAACCGCCTCCTGATCCAC |
| | CTCCGCTGCCACCCCCCGAACCTCCCTTGTACAGCTCGTCCATGCCGAGA |
| | GTG (SEQ ID NO: 17) |
| R7 | 5′-GCCTGACCCACCACCACTCCCGCCGCCGGAACCGCCCTTGTACAGCT |
| | CGTCCATGCCGAGAGTG (SEQ ID NO: 18) |
| R24 | 5′-GACCCACCACCACTCCCGCCGCCGGAACCGCCTCCTTGAAGGCCAAT |
| | GATGCCAC (SEQ ID NO: 19) |
| R25 | 5′-CGCTGCCACCCCCCGAACCTCCTCCTTGAAGGCCAATGATGCCAC |
| | (SEQ ID NO: 20) |
| R26 | 5′-GCCACTACCACCGCCACTTCCACCGCCGGACCCGCCCTTGTACAGCTC |
| | GTCCATGCCGAGAGTG (SEQ ID NO: 21) |
| R27 | 5′-GCCACTACCACCGCCACTTCCACCGCCGGACCCGCCTCCTTGAAGGC |
| | CAATGATGCCAC (SEQ ID NO: 22) |
| R37 | 5′-CTTTCGGGCTTTGTTAGCAGCCGGATCTCATCCTTGAAGGCCAATGAT |
| | GCCACATGC (SEQ ID NO: 23) |

Gene fragments

| | |
|---|---|
| G5 | GTGGGTCTGGGGGGGGATCCGGCGTGAGCAAGGGCGAGGAGCTGTTCA |
| (Sirius) | CCGGGGTGGTGCCCATCCTGGTCGAGCTGGACGGCGACGTAAACGGCCA |
| | CAGGTTCAGCGTGTCCGGCGAGGGCGAGGGCGATGCCACCTACGGCAAG |
| | CTGACCCTGAAGCTCATCTGCACCACCGGCAAGCTGCCCGTGCCCTGGC |
| | CCACCCTCGTGACCACCCTGCAATTCGGCGTGCTGTGCTTCGCCCGCTAC |
| | CCCGACCACATGAAGCAGCACGACTTCTTCAAGTCCGCCATGCCCGAAG |
| | GCTACGTCCAGGAGCGTACCATCTTCTTCAAGGACGACGGCAACTACAA |
| | GACCCGCGCCGAGGTGAAGTTCGAGGGCGACACCCTGGTGAACCGCATC |
| | GAGCTGAAGGGCATCGACTTCAAGGAGGACGGCAACATCCTGGGGCAC |
| | AAGCTGGAGTACAACGGGATAAGCTCAAACGTATATATCACCGCCGACA |
| | AGCAGAAGAACGGCATCAAGGCCCACTTCAAGATCCGCCACAACATCG |
| | AGGACGGCGGCGTGCAGCTCGCCGACCACTACCAGCAGAACACCCCCAT |
| | CGGCGACGGCCCCGTGCTGCTGCCCGACAACCACTACCTGAGCGTCCAG |
| | TCCAAGCTGAGCAAAGACCCCAACGAGAAGCGCGATCACATGGTCCTGC |
| | TGGAGTCCGTGACCGCCGCCGGGATCACTCTCGGCATGGACGAGCTGTA |
| | CAAGTGAGATCCGGCTGCTAACAAAGCCCGAAAG (SEQ ID NO: 24) |
| G10 | CGGGAGTGGTGGTGGGTCAGGCTCTGGCTGGTACACTTTTACGAAAAAT |
| (CAD4a) | ACCGCCATTAAATCCGCTGCCTCTGACTCCGCTAAAACCGTGGGTACATA |
| | TTCAAAGGGAAATCGTGTTTATTACAATGCGGAGATTACAACTAACGGC |
| | GAGACATGGCTTCGCTACCTTAGCTATTCGGGCTCTGAACACTTCGTGAA |
| | GATTGCGGCTGCTAAAACTACAACCACAAAGCCAGCTGCATCTACCTCT |
| | AAAACTGTTACCAAGAACGAGACAGGAACTTATAAATTCACCAAAACCA |
| | CGGCCATCAAGGGCTCGGTTTCGGATTCCGCCAAGACCCTGGGAACTTA |
| | CTACAAAGGGGACACCGTATATTATAATGCCAAAGTTACTAAAAATGGA |
| | GAGACGTGGCTTCGTTACCTGTCGTATTCCGGCGCTCAACACTATGTGAA |
| | GATTTCTGGCGCAGCCACGTCGACTACCACGACAAAACCCGCAACGTCT |
| | TCGAGTAAGACCGTCACGAAAGCTGAGACTGGCACATATAAATTTACAG |
| | GCACTACGGCTATTAAGGGCAGCGTGAACGATTCGGCCAAAACCTTAGG |
| | AACATACTATAAGGGGGACACCGTTTATTACAATGCGAAAGTTACAAAA |
| | AATGGGCAGACCTGGTTACGCTATTTGTCGTATTCCGGGGCACAGCACT |
| | ACGTGAAAATCTCTGGTTGAGATCCGGCTGCTAACAAAGCCCGAAAG |
| | (SEQ ID NO: 25) |
| G29 | ATGGCAAAGGGAGTGGCGGTACTGTCGTCCAGTGAAGGCGTCGCTGGGA |
| | CGATACTTTTTACACAGGAAGGTGATGGACCGACAACTGTCACTGGGAA |
| | CATTAGTGGACTGAAACCGGGATTACATGGGTTCCACGTCCATGCCCTTG |
| | GCGACACCACCAACGGATGTATGTCAACAGGACCACATTTCAATCCGGC |
| | GGGCAAAGAACATGGGTCCCCTGAGGACGAAACCCGCCATGCGGGCGA |
| (SOD) | CTTGGGTAACATCACGGTGGGAGATGACGGAACGGCGTGTTTTACTATC |
| | GTAGATAAACAGATTCCTTTAACCGGACCGCACTCAATTATCGGTCGGG |
| | CCGTCGTGGTGCACGCCGACCCGGACGACTTAGGCAAGGGGGGACACG |
| | AGCTTTCAAAATCTACGGGAAATGCTGGAGGTCGGATCGCATGTGGCAT |
| | CATTGGCCTTCAAGGA (SEQ ID NO: 26) |

### Example 2

Heterologous Display of Sirius-CAD4a on Lactic Acid Bacteria

Anchoring of Sirius-CAD4a to Lactic Acid Bacteria

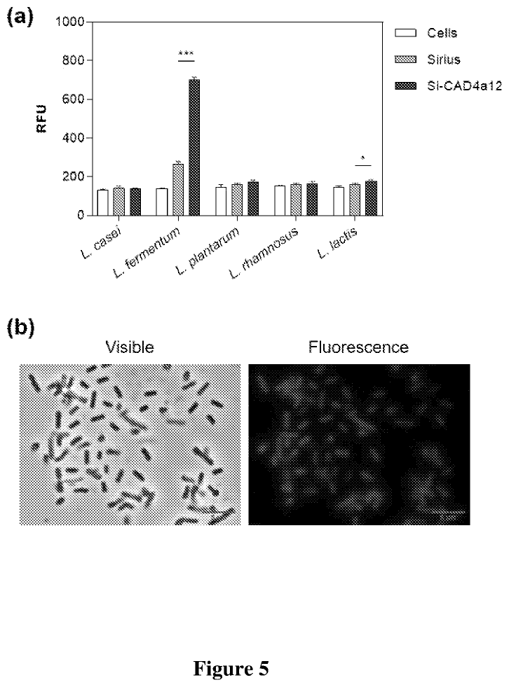

Lactobacillus casei, L. fermentum, L. plantarum, L. rhamnosus and Lactococcus lactis were grown to mid-log (OD₆₀₀ 0.8-1.2) or stationary phase (overnight culture), washed once with 10% glycerol, then resuspended in a 50:50 mix of MRS and 20% glycerol, aliquoted, and frozen for subsequent cell binding studies. Except where stated otherwise, the following protocol was used for the binding studies in this example. Log-phase cells were washed twice with binding buffer (1×PBS, pH 5), diluted to OD₆₀₀=1.5, and resuspended in 75 μl of binding buffer containing 2 μM of Sirius-CAD4a12. The mixture was incubated for 1.5 hr at 37° C. without shaking, then pelleted and washed twice with the same binding buffer before transfer to a black 96-well polystyrene plate for fluorescence measurement. Cell-associated fluorescence was measured on a spectrophotometer (Tecan, USA) with excitation at 355 nm and emission at 424 nm. The background fluorescence of the cells was subtracted to obtain a reading in relative fluorescence units (RFU). All studies were performed in triplicate. Cell imaging was performed on a Nikon Eclipse Ni-U microscope using a DAPI filter and 60×oil immersion lens. FIG. 5a shows the cell-associated fluorescence for all five LAB strains tested. There was at least a two-fold increase in cell-associated fluorescence on L. fermentum with the addition of CAD4a to Sirius. FIG. 5b shows the distribution of the fluorescent protein on L. fermentum, imaged using fluorescence microscopy.

### Example 3

Factors Influencing Binding of CAD4a to L. fermentum

Effect of Cell Pre-Treatment

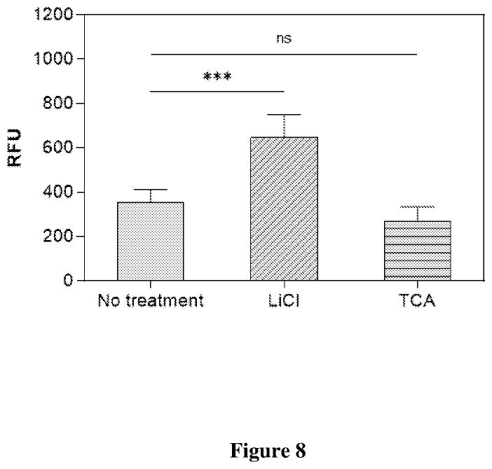

L. fermentum was treated with either 5 M LiCl or 10% v/v trichloroacetic acid (TCA) at 37° C. with shaking for 1 hr. Cells were washed twice with pH 7 PBS and once with pH 5 PBS before binding experiments (as above). TCA hydrolyses teichoic acids (TAs), one of the major cell wall components, whereas 5 M LiCl removes non-covalently bound surface proteins. As shown in FIG. 8, treatment with LiCl led to a two-fold increase in cell anchoring, as removal of surface proteins exposed more peptidoglycan binding sites for CAD4a. TCA treatment did not significantly impact CAD4a binding, thus the anchoring domain is not targeting cell wall TAs.

Influence of Cell Growth Phase, Salt Concentration, pH, and Binding Temperature

The effect of cell growth phase on CAD4a binding capacity was investigated with L. fermentum at mid-log and stationary phase. The influence of salt concentration on CAD4a binding to L. fermentum was tested using phosphate buffer (pH 5) supplemented with NaCl to final concentrations of 0.05, 0.1, 0.15, 0.2, 0.3, 0.4 M NaCl. The influence of pH was evaluated using PBS at pH 4.5, 5, 5.5, 6, 6.5, 7, 8 and 9. The effect of binding temperature was tested at 25° C., 30° C. and 37° C., with half-hourly timepoints to 3 hr. Protein concentration was 2 μM in these studies; protein binding and fluorescence measurement were carried out as described above.

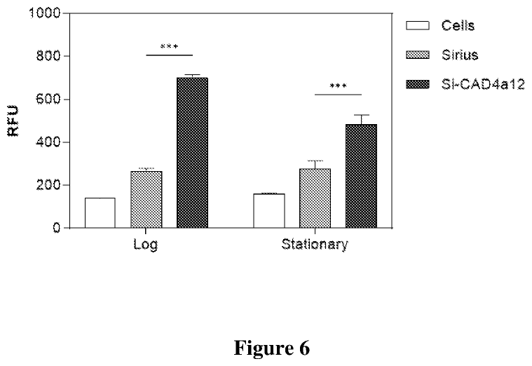

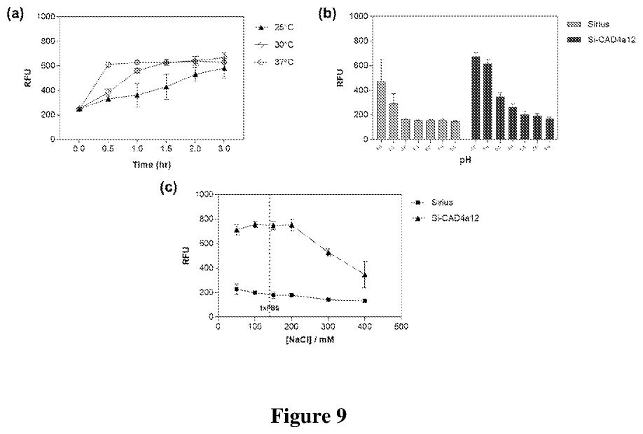

FIG. 6 compares surface display on log-phase and stationary-phase L. fermentum, showing less protein binding for the latter. FIG. 9a tracks CAD4a binding over 3 hr, showing that maximum binding was achieved within 1.5 hr at 30° C. and 37° C. FIG. 9b shows an optimal pH of 5 and below for CAD4a anchoring on L. fermentum. FIG. 9c shows that high salt concentrations (>200 mM NaCl) negatively impacted anchoring, likely by disrupting the non-covalent interactions between CAD4a and peptidoglycan.

### Example 4

Determination of Binding Capacity of CAD4a on L. fermentum

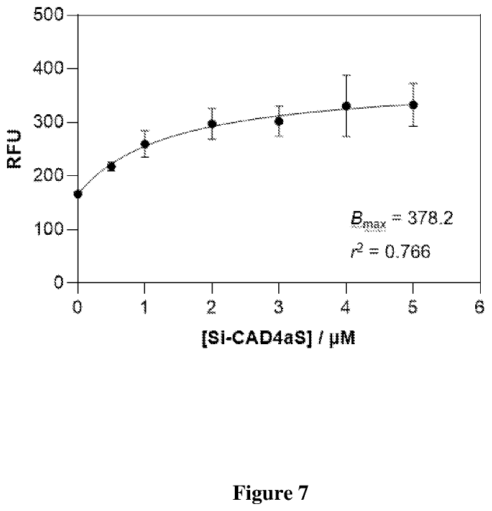

Fresh overnight cultures of L. fermentum were washed twice with binding buffer (pH 5 PBS), diluted to OD₆₀₀=1.5, and incubated with 70 μl of various concentrations of Sirius-CAD4a12 (0, 0.5, 1, 2, 3, 4, 5 μM). This was used to set up a standard curve to correlate RFU to protein concentration in the presence of cells. Subsequently, cell mixtures were pelleted and cell fluorescence determined as described above. All data points represent the average of at least three experiments. The Langmuir adsorption isotherm was assumed for the binding of CAD4a to the cell wall. Non-linear regression analysis was used to fit the binding data to the Langmuir model to determine Bₘₐₓ, the protein concentration at saturation. The binding capacity was calculated using Bₘₐₓ and the standard curve. FIG. 7 gives a saturation concentration of 1.05 μM protein for the given cell density (˜10⁹ cells/ml), or an average of 5×10⁵ molecules per cell.

### Example 5

Heterologous Display of SOD-CAD4a on L. fermentum, and Influence of Spacer Length on SOD Activity

Influence of Spacer Length on Activity of Surface-Displayed Superoxide Dismutase

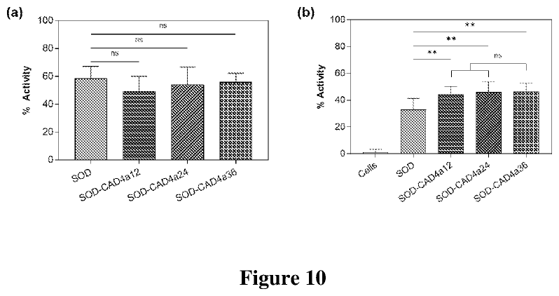

Superoxide dismutase (SOD) from Potentilla atrosanguinea was engineered with C-terminal CAD4a and three different spacer lengths (12-, 24- and 36-residues) between the enzyme and anchoring domain. Proteins were expressed in E. coli, and protein binding was performed with log-phase L. fermentum as described in Example 2. After washing, cells were resuspended in binding buffer for the SOD activity assay. The assay was performed using a commercial SOD kit (Sigma-Aldrich 19160) according to the manufacturer's protocol. The average gradient over the first 10 min (linear range) from triplicates was used to calculate the activity for each sample. FIG. 10a compares the activity of the three SOD variants, showing a dip in enzyme activity with the shortest spacer. All three spacer variants gave very similar activity after cell anchoring (FIG. 10b), thus spacer lengths of 12-36 residues had minimal effect on the protein functionality after anchoring.

### Example 6

Stability of Surface Display in Encapsulated Bacteria Under Simulated Gastric Digestion

Cell Encapsulation

Encapsulation was adapted from a previously-described protocol. Low-viscosity alginate (Sigma-Aldrich) was prepared as a 6% w/v stock in distilled water. Low molecular weight chitosan (Sigma-Aldrich) was dissolved in 0.1 M acetic acid to a concentration of 0.5% w/v, and the final pH adjusted to 5. Fresh overnight cultures of L. fermentum were washed twice with binding buffer (pH 5 PBS), diluted to OD₆₀₀=1.5, and incubated with 2 μM SOD-CAD4a36. After washing, 2 ml of cells was resuspended in 1.2 ml of 4% low-viscosity alginate (6% stock diluted with binding buffer) and mixed vigorously. The mixture was extruded dropwise into a 0.15 M CaCl₂ bath (pH 5) using a 21 G needle, and the beads were left to stir at room temperature for 1 hr, then rinsed once with binding buffer. The beads were subsequently added to 0.5% chitosan and left to stir for 10 min, then washed twice with binding buffer and kept at 4° C. till use. Empty beads and beads containing 1 μM SOD (without cells) were prepared as controls.

In Vitro Digestion

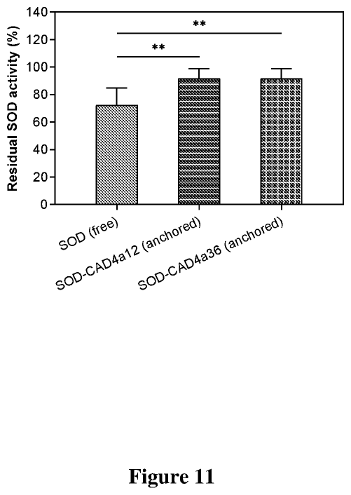

Simulated gastric fluid (SGF) was prepared as a 1× concentrate as previously described. Pepsin (Sigma-Aldrich P6887) and all necessary chemicals were purchased from Sigma-Aldrich. Sodium citrate was dissolved in PBS (pH 5) at a concentration of 0.15 M. Ten alginate-chitosan beads were used for each condition tested. Beads were suspended in 125 μl distilled water and equivolume of SGF, with additions of CaCl₂ (final concentration 0.075 mM) and pepsin (final concentration 0.5 mg/ml). The mixture was incubated with shaking at 37° C. for 2 hr, then rinsed twice with PBS (pH 7). Beads were incubated with pH 5 PBS as a control. The beads were dispersed in 250 μl citrate buffer held at 40° C., then centrifuged at 10,000 g for 10 min. The cell pellet was resuspended in 250 μl 1×PBS (pH 5) for the SOD assay. Residual activity represents cell-associated enzyme activity of SGF-treated beads relative to the PBS control. FIG. 11 shows that over 90% of cell-associated enzyme activity was retained after simulated gastric digestion, thus the polymer matrix offered substantial protection for the surface-displayed enzyme.

### Example 7

Heterologous Display of Sirius-CAD4a Variants Containing Different Number of SH3_5 Repeats

Expression of Si-CAD4a Variants with Different Number of SH3_5 Repeats

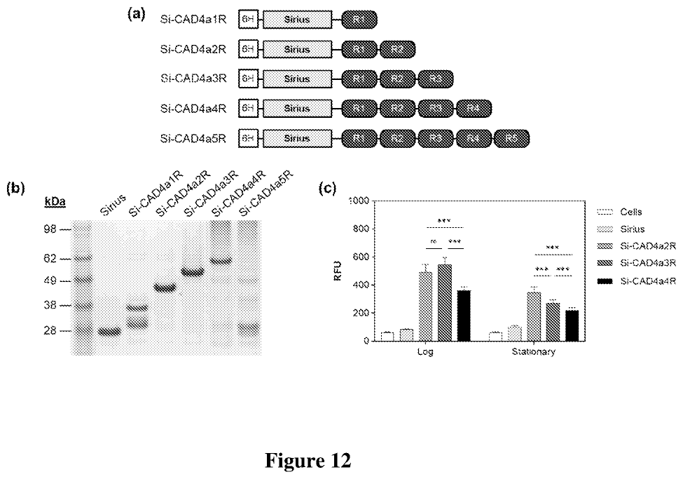

Variants of CAD4a containing one to five of the SH3_5 repeats in the Lys2 cell wall-targeting domain were constructed, all of which were conjugated to the Sirius fluorescent protein separated by a 12-residue spacer (FIG. 12a). These variants were expressed in E. coli and purified for analysis on SDS-PAGE gel. As shown in FIG. 12b, there was significant cleavage of the anchoring domain during expression of the one-repeat and five-repeat variants (CAD4a1R and CAD4a5R respectively). The yield of the latter was also significantly lower than for the other constructs. The two- and four-repeat variants (CAD4a2R, CAD4a4R) gave similar yields as the three-repeat variant (CAD4a3R) with minimal cleavage during expression.

Heterologous Display of Si-CAD4a Variants with Different Number of SH3_5 Repeats

The ability of the Si-CAD4a variants to anchor to L. fermentum in log and stationary growth phase (described in Example 2) were then compared. As shown in FIG. 12c, the lowest cell-associated fluorescence was observed for CAD4a4R, while CAD4a2R gave the best anchoring in stationary phase bacteria. Both CAD4a2R and CAD4a3R gave similarly good anchoring in log-phase cells.

1. An expression construct encoding a fusion protein, the expression construct comprising a nucleic acid encoding a first polypeptide for expression and a second polypeptide derived from the cell wall targeting region of a Lactobacillus plantarum Lys2 autolysin, wherein the first and second polypeptides are joined to form a fusion protein.

2. The expression construct of claim 1, wherein the first and second polypeptides are separated by a linker polypeptide.

3. The expression construct of claim 1, wherein the first polypeptide is positioned upstream of the second polypeptide.

4. The expression construct of claim 1, wherein the first polypeptide is positioned downstream of the second polypeptide.

5. The expression construct of claim 1, wherein the Lactobacillus autolysin comprises a cell wall targeting region.

6. The expression construct of claim 1, wherein the Lactobacillus autolysin comprises or consists of an amino acid sequence having at least 70% sequence identity to an amino acid sequence of SEQ ID NO: 1.

7. The expression construct of claim 1, wherein the second polypeptide comprises a SH3 type 5 motif polypeptide.

8. The expression construct of claim 1, wherein the second polypeptide comprises or consists of an amino acid sequence having at least 70% sequence identity to an amino acid sequence of SEQ ID NO: 2, SEQ ID NO: 3, SEQ ID NO: 4, SEQ ID NO: 5 and SEQ ID NO: 6, or combinations thereof.

9. The expression construct of claim 1, wherein the second polypeptide comprises or consists of an amino acid sequence having at least 70% sequence identity to an amino acid sequence of SEQ ID NO: 7.

10. The expression construct of claim 1, wherein the first polypeptide is superoxide dismutase (SOD).

11. The expression construct of claim 1, wherein the expression construct is comprised in a recombinant host cell, optionally the recombinant host cell is Escherichia coli or Lactococcus lactis.

12. (canceled)

13. The expression construct of claim 1, wherein the expression construct encodes a fusion protein.

14. A method of displaying a fusion protein on the surface of a lactic acid bacterium, the method comprising: a) expressing a fusion protein encoded by an expression construct in a host cell, and b) immobilising the fusion protein onto the surface of a lactic acid bacterium,
wherein the expression construct comprises a nucleic acid encoding a first polypeptide for expression and a second polypeptide derived from the cell wall targeting region of a Lactobacillus plantarum Lys2 autolysin, wherein the first and second polypeptides are joined to form a fusion protein.

15. The method of claim 14, wherein the method comprises isolating and/or purifying the fusion protein prior to b).

16. The method of claim 14, wherein the lactic acid bacterium is a Lactococcus or Lactobacillus bacterium.

17. The method of claim 14, wherein the lactic acid bacterium is Lactobacillus fermentum.

18.-21. (canceled)

22. A method of treating or preventing a condition or disease in a subject, the method comprising administering a lactic acid bacterium comprising a fusion protein encoded by an expression construct of claim 1 or a composition comprising a lactic acid bacterium comprising a fusion protein encoded by an expression construct of claim 1 to the subject.

23.-24. (canceled)

25. The method of claim 22, wherein the condition or disease is inflammatory bowel disease, oral mucositis, cancer, or an allergy.

26.-28. (canceled)

29. The method of claim 22, wherein the condition or disease is a pathogenic infection.


SEQUENCE LISTING (26 sequences)

SEQ ID NO: 1 - protein, 860 aa, Artificial Sequence
  L. plantarum Lys2 autolysin
MNFKSQNPIDASQSERFKLYKSGKLWLVAGLTFFSFLGGSVLNDTNVHADATNATTTSSSAASTASSASVATSSAASDETTKATSVSSSAATQVTSAATTKTTSQAATASSSAATSQSTSTASSSTSQATKTASSAATSTSATATSQATSVASSTATTTQVSHTASSTAASAASSTATVKAVKAASSTVSSAATTATKATAKVAAVAYTAATTSSTDVWTIGDTTRPRVDVVDVASYQSTMTQSDFNKLKAAGVKTVIVKSTEGTDYTNPAALNQAKMANKAGLNVDFYHYATFSTADAAKSEATNMAGFLVKNNVSTKVLLFADMEDSSSYSVNATANLNAFWSTLDSFGYKNHGVYTSNSYLYRDAVVKTVGQSRVWRAQYPYTPSANNLWNTNDGAWQFSDTALLPSGSDYTGYIDVSIDYNGLTEDSAGTNTFVTTTSSNDTTTSEVTTDTSATTNTSTSSTTKKASGWYTFTKNTAIKSAASDSAKTVGTYSKGNRVYYNAEITTNGETWLRYLSYSGSEHFVKIAAAKTTTTKPAASTSKTVTKNETGTYKFTKTTAIKGSVSDSAKTLGTYYKGDTVYYNAKVTKNGETWLRYLSYSGAQHYVKISGAATSTTTTKPATSSSKTVTKAETGTYKFTGTTAIKGSVNDSAKTLGTYYKGDTVYYNAKVTKNGQTWLRYLSYSGAQHYVKISGAATTTTTSSKSTATASAKTVAQSGTYKFAKTTAIKSSASDAASTVGTYYKGNTVNYNAKVTTNGQTWLRYTSYSGAQHYVKVSGGAATTTSSSASKTTAAAGTYTFKTTTNIRTAASLNASVVGQYSAGESVYYIGTVSADGYQWLKYLSNSGAYHYVAKIG

SEQ ID NO: 2 - protein, 82 aa, Artificial Sequence
  R1 of Lys2 CWT
SGWYTFTKNTAIKSAASDSAKTVGTYSKGNRVYYNAEITTNGETWLRYLSYSGSEHFVKIAAAKTTTTKPAASTSKTVTKNE

SEQ ID NO: 3 - protein, 84 aa, Artificial Sequence
  R2 of Lys2 CWT (SEQ ID NO 3)
TGTYKFTKTTAIKGSVSDSAKTLGTYYKGDTVYYNAKVTKNGETWLRYLSYSGAQHYVKISGAATSTTTTKPATSSSKTVTKAE

SEQ ID NO: 4 - protein, 84 aa, Artificial Sequence
  R3 of Lys2 CWT
TGTYKFTGTTAIKGSVNDSAKTLGTYYKGDTVYYNAKVTKNGQTWLRYLSYSGAQHYVKISGAATTTTTSSKSTATASAKTVAQ

SEQ ID NO: 5 - protein, 79 aa, Artificial Sequence
  R4 of Lys2 CWT
SGTYKFAKTTAIKSSASDAASTVGTYYKGNTVNYNAKVTTNGQTWLRYTSYSGAQHYVKVSGGAATTTSSSASKTTAAA

SEQ ID NO: 6 - protein, 61 aa, Artificial Sequence
  R5 of Lys2 CWT
GTYTFKTTTNIRTAASLNASVVGQYSAGESVYYIGTVSADGYQWLKYLSNSGAYHYVAKIG

SEQ ID NO: 7 - protein, 228 aa, Artificial Sequence
  CAD4a
SGWYTFTKNTAIKSAASDSAKTVGTYSKGNRVYYNAEITTNGETWLRYLSYSGSEHFVKIAAAKTTTTKPAASTSKTVTKNETGTYKFTKTTAIKGSVSDSAKTLGTYYKGDTVYYNAKVTKNGETWLRYLSYSGAQHYVKISGAATSTTTTKPATSSSKTVTKAETGTYKFTGTTAIKGSVNDSAKTLGTYYKGDTVYYNAKVTKNGQTWLRYLSYSGAQHYVKISG

SEQ ID NO: 8 - protein, 33 aa, Artificial Sequence
  Lys2 spacer
VTTTSSNDTTTSEVTTDTSATTNTSTSSTTKKA

SEQ ID NO: 9 - protein, 12 aa, Artificial Sequence
  Synthetic spacer
GGSGGGSGGGSG

SEQ ID NO: 10 - protein, 152 aa, Artificial Sequence
  SOD
MAKGVAVLSSSEGVAGTILFTQEGDGPTTVTGNISGLKPGLHGFHVHALGDTTNGCMSTGPHFNPAGKEHGSPEDETRHAGDLGNITVGDDGTACFTIVDKQIPLTGPHSIIGRAVVVHADPDDLGKGGHELSKSTGNAGGRIACGIIGLQG

SEQ ID NO: 11 - DNA, 30 bp, Artificial Sequence
  F1 primer
tgagatccggctgctaacaaagcccgaaag

SEQ ID NO: 12 - DNA, 58 bp, Artificial Sequence
  F15 primer
ggcgggtccggcggtggaagtggcggtggtagtggcggaggttcggggggtggcagcg

SEQ ID NO: 13 - DNA, 53 bp, Artificial Sequence
  F18 primer
ccacggtgggtctggggggggatccggcatggcaaagggagtggcggtactgt

SEQ ID NO: 14 - DNA, 34 bp, Artificial Sequence
  R1 primer
gccttggttttctaattttggttcaaagaaagct

SEQ ID NO: 15 - DNA, 23 bp, Artificial Sequence
  R4 primer
gccggatccccccccagacccac

SEQ ID NO: 16 - DNA, 30 bp, Artificial Sequence
  R5 primer
ctttcgggctttgttagcagccggatctca

SEQ ID NO: 17 - DNA, 100 bp, Artificial Sequence
  R6 primer
gcctgacccaccaccactcccgccgccggaaccgcctcctgatccacctccgctgccaccccccgaacctcccttgtacagctcgtccatgccgagagtg

SEQ ID NO: 18 - DNA, 64 bp, Artificial Sequence
  R7 primer
gcctgacccaccaccactcccgccgccggaaccgcccttgtacagctcgtccatgccgagagtg

SEQ ID NO: 19 - DNA, 55 bp, Artificial Sequence
  R24 primer
gacccaccaccactcccgccgccggaaccgcctccttgaaggccaatgatgccac

SEQ ID NO: 20 - DNA, 45 bp, Artificial Sequence
  R25 primer
cgctgccaccccccgaacctcctccttgaaggccaatgatgccac

SEQ ID NO: 21 - DNA, 64 bp, Artificial Sequence
  R26 primer
gccactaccaccgccacttccaccgccggacccgcccttgtacagctcgtccatgccgagagtg

SEQ ID NO: 22 - DNA, 59 bp, Artificial Sequence
  R27 primer
gccactaccaccgccacttccaccgccggacccgcctccttgaaggccaatgatgccac

SEQ ID NO: 23 - DNA, 57 bp, Artificial Sequence
  R37 primer
ctttcgggctttgttagcagccggatctcatccttgaaggccaatgatgccacatgc

SEQ ID NO: 24 - DNA, 767 bp, Artificial Sequence
  G5 (Sirius) gene fragment
gtgggtctggggggggatccggcgtgagcaagggcgaggagctgttcaccggggtggtgcccatcctggtcgagctggacggcgacgtaaacggccacaggttcagcgtgtccggcgagggcgagggcgatgccacctacggcaagctgaccctgaagctcatctgcaccaccggcaagctgcccgtgccctggcccaccctcgtgaccaccctgcaattcggcgtgctgtgcttcgcccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgggataagctcaaacgtatatatcaccgccgacaagcagaagaacggcatcaaggcccacttcaagatccgccacaacatcgaggacggcggcgtgcagctcgccgaccactaccagcagaacacccccatcggcgacggccccgtgctgctgcccgacaaccactacctgagcgtccagtccaagctgagcaaagaccccaacgagaagcgcgatcacatggtcctgctggagtccgtgaccgccgccgggatcactctcggcatggacgagctgtacaagtgagatccggctgctaacaaagcccgaaag

SEQ ID NO: 25 - DNA, 736 bp, Artificial Sequence
  G10 (CAD4a) gene fragment
cgggagtggtggtgggtcaggctctggctggtacacttttacgaaaaataccgccattaaatccgctgcctctgactccgctaaaaccgtgggtacatattcaaagggaaatcgtgtttattacaatgcggagattacaactaacggcgagacatggcttcgctaccttagctattcgggctctgaacacttcgtgaagattgcggctgctaaaactacaaccacaaagccagctgcatctacctctaaaactgttaccaagaacgagacaggaacttataaattcaccaaaaccacggccatcaagggctcggtttcggattccgccaagaccctgggaacttactacaaaggggacaccgtatattataatgccaaagttactaaaaatggagagacgtggcttcgttacctgtcgtattccggcgctcaacactatgtgaagatttctggcgcagccacgtcgactaccacgacaaaacccgcaacgtcttcgagtaagaccgtcacgaaagctgagactggcacatataaatttacaggcactacggctattaagggcagcgtgaacgattcggccaaaaccttaggaacatactataagggggacaccgtttattacaatgcgaaagttacaaaaaatgggcagacctggttacgctatttgtcgtattccggggcacagcactacgtgaaaatctctggttgagatccggctgctaacaaagcccgaaag

SEQ ID NO: 26 - DNA, 456 bp, Artificial Sequence
  G29 (SOD) gene fragment
atggcaaagggagtggcggtactgtcgtccagtgaaggcgtcgctgggacgatactttttacacaggaaggtgatggaccgacaactgtcactgggaacattagtggactgaaaccgggattacatgggttccacgtccatgcccttggcgacaccaccaacggatgtatgtcaacaggaccacatttcaatccggcgggcaaagaacatgggtcccctgaggacgaaacccgccatgcgggcgacttgggtaacatcacggtgggagatgacggaacggcgtgttttactatcgtagataaacagattcctttaaccggaccgcactcaattatcggtcgggccgtcgtggtgcacgccgacccggacgacttaggcaaggggggacacgagctttcaaaatctacgggaaatgctggaggtcggatcgcatgtggcatcattggccttcaagga